(19) United States
(12) Patent Application Publication
(10) Pub. No.: US 2011/0144028 A1
(43) Pub. Date: Jun. 16, 2011

(54) MODULATING CELLULAR ELECTROPHYSIOLOGY

(76) Inventor: Vinod Sharma, Blaine, MN (US)

(21) Appl. No.: 12/636,312

(22) Filed: Dec. 11, 2009

(51) Int. Cl. A61K 38/16; C12N 5/071; A61P 9/00; A61K 31/4439

(52) U.S. Cl. 514/17.4; 435/325; 514/341; 514/355

(57) ABSTRACT

The present invention relates to compositions, apparatus, and methods for improving the viability of cells, including, but not limited to, nonexcitable cells, and tissues expressing exogenous polynucleotides that encode membrane proteins that regulate that flow of ions across the cell membrane. The viability of the cells and tissues may be improved by contacting the cells or tissue with one or more ion channel blocking agents. Membrane proteins that regulate the flow of ions across the cell membrane include, but are not limited to, ion channels.

### BACKGROUND

Ion channels are pore-forming proteins that help establish and control the small voltage gradient across the plasma membrane of all living cells by allowing the flow of ions down their electrochemical gradient. They are present in the membranes that surround all biological cells and regulate the flow of ions across the membrane. Ion channels are key components in a wide variety of biological processes that involve rapid changes in cells, such as cardiac, skeletal, and smooth muscle contraction, epithelial transport of nutrients and ions, T-cell activation and pancreatic beta-cell insulin release. Genetic engineering approaches that result in the expression of exogenous ion channels have therapeutic potential to modify cellular electrophysiology and automaticity.

The heart is an electromechanical organ with considerable electrical heterogeneity and this electrical heterogeneity is conferred by the differential expression of a variety of ion channels in various regions of the heart. The expression of exogenous ion channels to change the underlying electrophysiology of the heart is an attractive therapeutic approach to modify cardiac cellular electrophysiology in variety of cardiac dysfunctions, including, but not limited to, ventricular tachycardia, arrhythmia, atrial fibrillation, bradycardia, and heart failure. Whether such ion channel therapies present considerable therapeutic potential, the complete impact of the changes that an externally introduced ion channel confers in a heterogeneous cell population, including, but not limited to, cardiac tissue, is not understood.

The present invention presents a method of increasing the viability of cells and tissues expressing exogenous nucleotide sequences that encode membrane polypeptides regulating the flow of ions across the cell membrane.

### SUMMARY OF THE INVENTION

The present invention includes a method of increasing the viability of cells expressing an exogenous polynucleotide encoding a membrane polypeptide that regulates the flow of ions across a cell membrane, the method including contacting the cells expressing the exogenous polynucleotide with an ion channel blocking agent. In some embodiments, the membrane polypeptide that regulates the flow of ions across a cell membrane is not a hyperpolarized activated cyclic nucleotide (HCN) channel. In some embodiments, the membrane polypeptide that regulates the flow of ions across a cell membrane is an ion channel. In some embodiments, the cells comprise nonexcitable cells. In some embodiments, the cells comprise excitable cells. In some embodiments, the viability of nonexcitable cells in the heterologous tissue is improved.

The present invention includes a method of increasing the viability of nonexcitable cells in a heterologous tissue expressing an exogenous polynucleotide encoding a membrane polypeptide that regulates the flow of ions across a cell membrane, the method including contacting the cells expressing the exogenous polynucleotide with an ion channel blocking agent. In some embodiments, the membrane polypeptide that regulates flow of ions across a cell membrane is an ion channel. In some embodiments, the membrane polypeptide that regulates the flow of ions across a cell membrane is not a hyperpolarized activated cyclic nucleotide (HCN) channel.

The present invention includes a method of increasing the viability of cells in a heterologous tissue expressing an exogenous polynucleotide encoding a membrane polypeptide that regulates the flow of ions across a cell membrane, the method including contacting the cells expressing the exogenous polynucleotide with an ion channel blocking agent. In some embodiments, the membrane polypeptide that regulates flow of ions across a cell membrane is an ion channel. In some embodiments, the membrane polypeptide that regulates the flow of ions across a cell membrane is not a hyperpolarized activated cyclic nucleotide (HCN) channel.

The present invention includes a method of modifying the electrophysiological function of a heterologous tissue including excitable cells and nonexcitable cells, the method including contacting the heterologous tissue with one or more ion channel blocking agents before, after, and/or coincident to transfection or transduction of the heterologous tissue with an exogenous polynucleotide encoding a membrane polypeptide that regulates the flow of ions across a cell membrane. In some embodiments, the membrane polypeptide that regulates flow of ions across a cell membrane is an ion channel. In some embodiments, the viability of nonexcitable cells in the heterologous tissue is improved. In some embodiments, the membrane polypeptide that regulates the flow of ions across a cell membrane is not a hyperpolarized activated cyclic nucleotide (HCN) channel.

The present invention includes a method of modifying the electrophysiological function of a heterologous tissue including excitable cells and nonexcitable cells, the method including transfecting or transducing said heterologous tissue with an exogenous polynucleotide encoding a membrane polypeptide that regulates the flow of ions across a cell membrane; and contacting said heterologous tissue with an ion channel blocking agent. In some embodiments, the membrane polypeptide that regulates the flow of ions across a cell membrane is not a hyperpolarized activated cyclic nucleotide (HCN) channel. In some embodiments, the membrane polypeptide that regulates flow of ions across a cell membrane is an ion channel. In some embodiments, the heterologous tissue is contacted with an amount of ion channel blocking agent effective to increase viability of the nonexcitable cells within the heterologous tissue.

In some embodiments of the methods of the present invention, a heterologous tissue includes excitable cells and nonexcitable cells. In some embodiments, the method increases the viability of nonexcitable cells. In some embodiments, the method increases the viability of excitable cells. In some embodiments, the heterologous tissue is cardiac tissue.

With the methods of the present invention, cells or heterologous tissue may be contacted with the ion channel blocking agent before, after, and/or coincident to the expression of the polynucleotide encoding the polypeptide.

With the methods of the present invention, an ion channel blocking agent may be formulated in composition for controlled or sustained release.

With the methods of the present invention, an ion channel includes a gap junction channel, calcium channel, a sodium channel, a chloride channel, a hyperpolarized activated cyclic nucleotide (HCN) channel, SERCA2a, a non-specific leak channel, or a potassium channel. In some embodiments, the ion channel includes a potassium channel. In some embodiments, the potassium channel includes a member of the Kv1-9 family. In some embodiments, the potassium channel includes Kv1.3. In some embodiments, the membrane polypeptide that regulates the flow of ions across a cell membrane is not a hyperpolarized activated cyclic nucleotide (HCN) channel.

With the methods of the present invention, the ion channel blocking agent includes a calcium channel blocking agent, a sodium channel blocking agent, a gap junction channel blocking agent, a chloride channel blocking agent, a hyperpolarized activated cyclic nucleotide (HCN) channel blocking agent, a SERCA2a blocking agent, a non-specific leak channel blocking agent, and/or a potassium channel blocking agent. In some embodiments, the ion channel blocking agent comprises a potassium channel blocking agent. In some embodiments, the ion channel blocking agent comprises charybdotoxin. In some embodiments, the ion channel blocking agent is not a hyperpolarized activated cyclic nucleotide (HCN) channel blocking agent.

With the methods of the present invention, contacting the cells or the heterologous tissue includes intermittent and/or continuous delivery of the ion channel blocking agent.

The present invention includes a composition including an ion channel blocking agent, wherein the composition is in a formulation for controlled or sustained release.

The present invention includes a kit including an ion blocking agent and a delivery device for delivery of the ion blocking agent to a cell or tissue expressing an exogenous polynucleotide encoding a membrane polypeptide that regulates the flow of ions across a cell membrane.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows electrical heterogeneity of various regions of the heart.

FIG. 2 shows Kv1.3 as a model ion channel.

FIG. 3 shows viability of cardiac myocytes in the presence of Kv1.3. In FIG. 3A, brightfield imaging confirms expression of AdV-Kv1.3-GFP. In FIG. 3B, fluorescent imaging reveals non-apoptotic

FIG. 4 shows that Kv1.3 induces degradation of cardiac fibroblasts by blebbing, rounding, and detachment. In FIG. 4A, brightfield imaging confirms expression of AdV-Kv1.3-GFP. In FIG. 4B, Hoechst dye staining reveals apoptotic nuclei as indicated by the arrows.

FIG. 5 shows dose-dependent reduction of Cx43. In FIG. 5A, immunofluorescence shows expression of both α-actinin and Cx43 in non-transfected cardiac myocytes. In FIG. 5B, fluorescent imaging of Cx43 alone confirms normal expression in the non-transfected myocytes. In FIG. 5C, fluorescent imaging of myocytes transfected with 10⁸ PFU/ml Ad-Kv1.3-GFP confirms expression of Kv1.3 and shows continued α-actinin expression. Expression of Cx43 is highly disrupted. In FIG. 5D, fluorescent imaging of Cx43 alone confirms reduced and highly disrupted expression of Cx43 in Kv1.3 expressing myocytes (compare to FIG. 5B).

FIG. 6 shows deterioration of cardiac fibroblasts in a dose-dependent manner. In FIGS. 6A-D, phase contrast microscopy shows cell morphology and decreasing number of fibroblasts with increasing Kv1.3. In FIGS. 6E-H, fluorescence microscopy shows expression of Kv1.3 following transfection with AdV-Kv1.3-GFP. FIGS. 6A and 6E are non-transfected control fibroblasts. Increased Kv1.3 (FIGS. 6E-H) correlates with a loss of cell number and disruption in cellular morphology (FIGS. 6B-D).

FIG. 7 shows an increase in apoptosis in cardiac fibroblasts in a dose-dependent manner. FIG. 7A represents the addition of Charybdotoxin alone and FIG. 7B represents the addition of Charybdotoxin and 5% serum results in partial rescue of cells from Kv1.3 induced apoptosis.

FIG. 8 shows cell viability is rescued in cardiac fibroblasts expressing Kv3.1 with the addition of Charybdotoxin. In FIGS. 8A and 8B, phase contrast microscopy shows cellular morphology of the fibroblasts. In FIGS. 8C and 8D, fluorescent microscopy confirms expression of Kv1.3 following transfection with 10⁷ PFU/ml AdV-Kv1.3-GFP. FIG. 8A shows Kv1.3 induces degradation of cardiac fibroblasts by blebbing, rounding, and detachment. FIG. 8B shows the addition of Charbdyotoxin restores normal fibroblast morphology.

### DETAILED DESCRIPTION OF ILLUSTRATIVE EMBODIMENTS OF THE PRESENT INVENTION

The present invention relates to compositions, apparatus, and methods for improving the viability of cells and tissues expressing exogenous polynucleotides encoding membrane proteins that regulate the flow of ions across the cell membrane. The viability of the cells and tissues is improved by contacting the cells or tissue with one or more ion channel blocking agents. Membrane proteins that regulate the flow of ions across the cell membrane, which are encoded by an exogenously administered polynucleotide include, but are not limited to, transmembrane ion channel proteins, active ion transport proteins and ion exchange proteins.

In some embodiments of the present invention, a membrane protein that regulates the flow of ions across the cell membrane is an ion channel protein. Ion channels regulate the flow of ions across the membrane in all cells and are integral membrane proteins. Typically, an ion channel is an assembly of several proteins. Such “multi-subunit” assemblies usually involve a circular arrangement of identical or homologous proteins closely packed around a water-filled pore through the plane of the membrane or lipid bilayer. The pore-forming subunit(s) are called the α-subunit, while the auxiliary subunits are denoted β, γ, and so on. While some channels permit the passage of ions based solely on charge, the archetypal channel pore conducts a specific species of ion, such as sodium or potassium, and conveys them through the membrane single file, nearly as quickly as the ions move through free fluid.

Ion channels of the present invention include voltage-gated ion channels, which activate/inactivate depending on the voltage gradient across the plasma membrane. Voltage-gated ion channels include, but are not limited to, voltage-gated sodium channels, voltage-gated calcium channels, voltage-gated potassium channels (KV), hyperpolarization-activated cyclic nucleotide-gated channels (HCN), and voltage-gated proton channels.

With some embodiments of the present invention, the ion channel is not a hyperpolarization-activated cyclic nucleotide-gated (HCN) channel.

Ion channels of the present invention include ligand-gated ion channels, which activate/inactivate depending on binding of a ligand to the channel. Ligand-gated ion channels, also known as ionotropic receptors, open in response to specific ligand molecules binding to the extracellular domain of the receptor protein. Ligand binding causes a conformational change in the structure of the channel protein that ultimately leads to the opening of the channel gate and subsequent ion flux across the plasma membrane. Examples of such channels include the cation-permeable “nicotinic” acetylcholine receptor, ionotropic glutamate-gated receptors and ATP-gated P2X receptors, and the anion-permeable y-aminobutyric acid-gated GABA_{A} receptor.

Ion channels of the present invention include chloride ion channels, sodium ion channels, calcium ion channels, proton channels, and potassium ion channels. Ion channels of the present invention include general ion channels that are relatively non-specific for ions and thus let many types of ions through the channel.

A potassium ion channel of the present invention may belong to any of the following major classes of ion channels. A calcium-activated potassium channel, which is open in response to the presence of calcium ions or other signaling molecules, also known as a Ca2⁺-activated K⁺ channel. An inwardly rectifying potassium channel passes current (positive charge) more easily in the inward direction, into the cell. A tandem pore domain potassium channel, which is constitutively open or possess high basal activation, such as the “resting potassium channels” or “leak channels” that set the negative membrane potential of cardiac cells. When open, they allow potassium ions to cross the membrane at a rate which is nearly as fast as their diffusion through bulk water. Such “leak” K⁺ channels are so termed because of the apparent lack of gating control. Or, a voltage-gated potassium channel, which opens or closes in response to changes in the transmembrane voltage.

A potassium ion channel of the present invention may be one of a large family of mammalian potassium channels, such as for example, Kv1 (shaker), Kv2, Kv3 (Shaw), Kv4 (Sha1), Kv5, Kv6, Kv7, Kv8, or Kv9. In one embodiment, the potassium ion channel is Kv1.3.

With the present invention, any of a wide variety of available ion channel blocking agents may be administered. An ion channel blocking agent may inhibit the movement of ions through the ion channels. As used herein, unless the context makes clear otherwise, “blocking” or “block” of an ion channel means any block or inhibition of current through that ion channel.

An ion channel blocking agent includes, but is not limited to, a calcium channel blocking agent, a sodium channel blocking agent, a gap junction channel blocking agent, a chloride channel blocking agent, a hyperpolarized activated cyclic nucleotide (HCN) channel blocking agent, a SERCA2a blocking agent, a non-specific leak channel blocking agent, and/or a potassium channel blocking agent.

In some embodiments of the present invention, the ion channel blocking agent is not a hyperpolarized activated cyclic nucleotide (HCN) channel blocking agent.

A sodium channel blocking agent inhibits the movement of sodium ions through a sodium channel. Examples of sodium channel blockers include, but are not limited to, tetrodotoxin (TTX) (used by puffer fish and some types of newts for defense), saxitoxin (produced by a dinoflagellate, also known as red tide), lidocaine and novocaine (local anesthetics which block sodium ion channels), quinidine, procainamide, disopyramide, phenytoin, mexiletine, tocainide, encainide, flecainide, propafenone, and moricizine.

A calcium channel blocking agent inhibits the movement of calcium ions through calcium channels. Examples include, but are not limited to, amlodipine, aranidipine, azelnidipine, barnidipine, benidipine, cilnidipine, clevidipine, diltiazin, efonidipine, felodipine, gallopamil, lacidipine, lercanidipine, manidipine, nicardipine, nifedipine, nilvadipine, nimodipine, nisoldipine, nitrendipine, pranidipine, and verapamil.

A chloride channel blocking agent inhibits the movement of chloride ions through chloride channels. Examples of sodium channel blockers include, but are not limited to, 4,4′-diisothiocyanatosilbene (DIDS), 4-acetamido-4′-isothiocyanatostilbene-2,2′-disulfonic acid (SITS), N-phenylanthracillic acid (NPA), g-amino-camptothecin (9-AC), 5-nitro-2-(3-phenylpropylamino)-benzoate (NPPB), flufenamate and diphenylamine-2-carboxylate (DPC), and niflumic acid.

Hyperpolarization-activated cyclic nucleotide-gated channels (HCN) serve as ion channels across the plasma membrane of heart and brain cells and are sometimes referred to as “pacemaker channels” because they help to generate rhythmic activity within groups of heart and brain cells. HCN channels are encoded by four genes (HCN1-4) and are widely expressed throughout the heart and the central nervous system. Examples of HCN channel blocking agents include, but are not limited to, ivabradine, L-cis diltiazem, tetracaine, calmodulin antagonists, 6-(phenylamino)-5,8-quinolinedone (LY83583), H-8 [N-2-(methylamino)ethyl-5-isoquinolinesulfonamide, and cesium chloride (CsCl).

A potassium channel blocking agent that inhibits the movement of potassium ions through the potassium channels. Examples include, but are not limited to, charydotoxin (a 37 amino acid neurotoxin from the venom of the scorpion Leiurus quinquestriatus hebraeus), dendrotoxin (produced by mamba snakes), iberiotoxin (produced by the Buthus tamulus), heteropodatoxin (produced by Heteropoda venatoria), amiodarone, bretylium, sotalol, dofetilide, sotalol, ibutilide, azimilide, clofilium, nifekalant, tedisamil, and sematilide.

With the present invention, the administration of an ion blocking agent increases the viability of cells expressing an exogenous polynucleotide encoding an ion channel. Cells, tissues, and organs may be contacted with one or more ion blocking agents before, after, and/or coincident to the expression of an exogenous polynucleotide encoding a membrane protein that regulates the flow of ions.

A polynucleotide may also be referred to herein as “polynucleotide sequence,” “nucleic acid,” “nucleic acid sequence,” “nucleotide sequence,” and similar terms. As used herein, the terms “encodes,” “encoding,” “coding sequence,” and similar terms refer to a nucleic acid sequence that is transcribed (in the case of DNA) and translated (in the case of mRNA) into a polypeptide in vitro or in vivo when placed under control of appropriate regulatory sequences.

Expression includes the transcription of a DNA sequence into a mRNA. Expression by also include the translation of such a transcribed mRNA into a polypeptide. Expression may be accomplished by a variety of methods, including, but not limited to, expression by transfection or transduction procedures. Transfection and transduction refer to the acquisition by a cell of new genetic material by incorporation of added nucleic acid molecules. Transfection can occur by physical or chemical methods. Transduction refers to the process of transferring nucleic acid into a cell using a DNA or RNA virus.

Ion channels of the present invention include full-length wild-type channels and functional variants or fragments thereof An ion channel may be of mammalian origin. In various embodiments, the ion channel may be of human origin.

The nucleic acid (e.g., cDNA or genomic DNA) encoding a membrane protein, including an ion channel, may be inserted into a vector for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to those of skill in the art.

A vector including a polynucleotide encoding an ion channel can be delivered into a cell by, for example, transfection or transduction procedures. Transfection and transduction refer to the acquisition by a cell of new genetic material by incorporation of added nucleic acid molecules. Transfection can occur by physical or chemical methods. Many transfection techniques are well known to those of ordinary skill in the art including, without limitation, calcium phosphate DNA co-precipitation, DEAE-dextrin DNA transfection, electroporation, naked plasmid adsorption, and cationic liposome-mediated transfection.

Transduction refers to the process of transferring nucleic acid into a cell using a DNA or RNA virus. Suitable viral vectors for use as transducing agents include, but are not limited to, retroviral vectors, lentiviral vectors, adenoviral vectors, adeno-associated viral vectors, vaccinia viral vectors, and Semliki Forest viral vectors.

Expression vectors containing DNA encoding an ion channel may be administered in vivo in any known or future developed manner. In various embodiments, the expression vectors are packaged into viruses, such as adenoviruses, and are delivered in proximity to targeted cells, tissue or organs. In various embodiments, the expression vectors are packaged into adenoviruses, such as helper-dependent adeno viral vector (HDAd) or adeno-associated virus pseudo-type 9 (AAV2/9). HDAd virus packaging typically elicits less of an immunogenic response in vivo compared to some other adenoviruses and thus allows for longer teen expression. AAV2/9 packaging can result in cardiac tropism as well as a prolonged expression time frame. Other viruses of clinical relevance include lentiviruses. Replication deficient lentiviruses are RNA viruses, which can integrate into the genome and lead to long-term functional expression. Viral vectors systems in addition to lentiviral vectors, AAV vectors, and HD AdV may also be used for the delivery of a polynucleotide encoding an ion channel. Alternatively, non-viral delivery systems may be employed. For example, liposomes, DNA complexes, plasmid, liposome complexes, naked DNA, DNA-coated particles, or polymer based systems may be used to deliver the desired sequence to the cells.

An exogenous nucleotide sequence encoding an ion channel may be administered to cardiac cells, such as cardiomyocytes, Purkinje cells, or conductive tissue, SAN or AVN, cardiac fibroblasts, or generally to the heart or portions thereof. An exogenous nucleotide sequence encoding an ion channel may be administered to non-cardiac cells, such as, for example, skeletal, and smooth muscle cells, epithelial cells, and nerve cells. Delivery of a nucleotide sequence encoding an ion channel to myocardial cells, such as, for example, cardiac atrial cells, Purkinje fiber cells or ventricular cells, can be carried out according to any method known or developed in the art. For example, the delivery or administration may be accomplished by injection, catheter and other delivering vehicle known or developed in the art.

Exogenous polynucleotides encoding membrane proteins regulating the flow of ions across a cell membrane, including, but not limited to, ion channels, can be made by traditional PCR-based amplification and known cloning techniques. Alternatively, a polynucleotide of the invention can be made by automated procedures that are well known in the art. A polynucleotide of the invention may include a start codon to initiate transcription and a stop codon to terminate translation. Suitable polynucleotides for use with the invention can be obtained from a variety of public sources including, without limitation, GenBank (National Center for Biotechnology Information (NCBI)), EMBL data library, SWISS-PROT (University of Geneva, Switzerland), the PIR-International database; and the American Type Culture Collection (ATCC).

With the present invention, ion channel blockers may be used to increase the viability of cells, tissues, and organs that express an exogenous polynucleotide that encodes a membrane polypeptide that regulates the flow of ions across the cell membrane. Cell viability may be monitored by any of a variety of methods, including, but not limited to, any of those described herein. The methods of the present invention may increase the viability of nonexcitable and/or non-excitable cells. In some embodiments, the viability of fibroblast is improved. In some embodiments, the viability of myocytes is improved. In some embodiments, the viability of fibroblasts and myocytes is improved.

With the present invention ion channel blockers may be used to prevent apoptosis in cells that express an exogenous polynucleotide that encodes a membrane polypeptide that regulates the flow of ions across the cell membrane. The extent of apoptosis in cells may be monitored by any of a variety of methods, including, but not limited to, any of those described herein. The methods of the present invention may prevent apoptosis in nonexcitable and/or non-excitable cells. In some embodiments, apoptosis in fibroblast is prevented. In some embodiments, apoptosis of myocytes is prevented. In some embodiments, apoptosis in fibroblasts and myocytes is prevented.

With the present invention, ion channel blockers may be used to modulate the electrophysiological function of cells, tissues, and organs that express an exogenous polynucleotide that encodes a membrane polypeptide that regulates the flow of ions across the cell membrane. Modulating means that the activity of the flow of ions across the cell membrane in the cells or tissues may be either increased or decreased. Electrophysiological function may be assayed by any of a variety of methods including, but not limited to, voltage clamp electrophysiology (in particular patch clamp), immunohistochemistry, histological analysis of a biopsy sample, and RT-PCR.

Membrane proteins that regulate the flow of ions across the cell membrane, including ion channels, play a vital role in the function of many cell and tissue types and have been implicated in a long list of diseases, including, for example, cystic fibrosis, diabetes, cardiac arrhythmias, neurologic and psychiatric diseases, gastrointestinal disorders, and hypertension. The methods of the present invention may be applied to any of a variety of cells, including, but not limited to, cardiac myocytes, fibroblasts, neuronal cells, skeletal muscle cells, smooth muscle cells, epithelial cells, endothelial cells, and immune cells, such as T lymphocytes. Such cells may be excitable cells or nonexcitable cells, wherein an excitable cell demonstrates electrical activity and a nonexcitable cell demonstrates no obvious electrical activity. The methods of the present invention may be applied to any of a variety tissue, including, but not limited to cardiac tissue, nervous tissue, skeletal muscle, smooth muscle, secretory epithelial tissue, and beta cells of the pancreas. A tissue may be heterologous tissue, including both excitable cells and nonexcitable cells. The methods of the present invention may affect the excitable cells and/or the nonexcitable cells within a heterologous tissue. Some embodiments of the present invention may affect fibroblasts and/or myocytes within a heterologous tissue.

The methods of the present invention may be applied to any of a variety of organs, including, but not limited to the heart, the brain, the spine, nerves, lung, bladder, and blood vessels, including veins and arteries.

As used herein, unless the context makes clear otherwise, “treatment,” and similar word such as “treated,” “treating” etc., is an approach for obtaining beneficial or desired results, including and preferably clinical results. Treatment can involve optionally either the amelioration of symptoms of the disease or condition, or the delaying of the progression of the disease or condition. As used herein, an “effective amount” or a “therapeutically effective amount” of a substance is that amount sufficient to affect a desired biological effect, such as beneficial results, including clinical results. In some embodiments of the methods of the present invention, more than one ion channel blocker may be administered.

The present invention includes compositions of ion channel blockers. A composition may also include, for example, buffering agents to help to maintain the pH in an acceptable range or preservatives to retard microbial growth. Such compositions may also include a pharmaceutically acceptable carrier. As used herein, the term “pharmaceutically acceptable carrier” refers to one or more compatible solid or liquid filler, diluents or encapsulating substances which are suitable for administration to a human or other vertebrate animal. The compositions of the present invention are formulated in pharmaceutical preparations in a variety of forms adapted to the chosen route of administration. A composition of the present invention may include a mixture or cocktail of two, three, four, five, or more blockers.

With the methods of the present invention, one or more additional therapeutic agents may be administered, in addition to the administration of an ion channel blocker. An ion channel blocker may be administered before, after, and/or coincident to the administration of one or more additional therapeutic agents. An ion channel blocker and one or more additional therapeutic agents may be administered separately or as a part of a mixture or cocktail.

The agents of the present invention can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or the injection or application into or around the site of the exogenously expressed polynucleotide encoding a membrane protein that regulates the flow of ions across the cell membrane.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, intracardiac, and intrapericardial administration. In this connection, sterile aqueous media that can be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparation may be pyrogen-free.

For enteral administration, the inhibitor may be administered in a tablet or capsule, which may be enteric coated, or in a formulation for controlled or sustained release.

Ion channel blockers may be formulated for controlled or sustained release locally at the site of the cells or tissue that are to express exogenous polynucleotides encoding a membrane proteins that regulates the flow of ions across the cell membrane. Many suitable formulations are known, including polymeric or protein microparticles encapsulating the ion blocking agent, gels, or solutions which can be used locally to administer drug. These can take the form of implants. Such an implant may be implanted within the tissue that is to express the exogenous polynucleotide encoding a membrane protein. Delivery of a blocker may also be via an osmotic implanted at the delivery site. Such a pump may be programmed to deliver a predetermined rate.

Delivery of an ion channel blocker may be acute (as a single administration), subacute (delivered over days or weeks), or a long term, chronic delivery. Delivery may be continuous or may be intermittent. Intermittent delivery may occur at predetermined timed intervals over the entire twenty-four hour day, for example, once a day, twice a day, three times a day, four times a day, six times a day, eight times a day, twelve times a day, or twenty-four times a day. A baseline dosage delivered continuously or intermittently at specified intervals, may be supplemented with a bolus dosage. Such a bolus dosage may be delivered in response to the determination of a difference between a currently measured physiological state in a subject and a baseline physiologic state.

In some embodiments of the present invention, an ion channel blocking agent may be delivered to the heart. Any of the wide variety of mechanisms for delivering to the heart available to one skilled in may be used, ranging, for example, from a simple injection, to the use of a catheter, to the use of a drug pump.

Such a pump may be, for example, an external drug pump, such as, for example, the Medtronic® MiniMed® pump, an implantable drug pump, such as, for example, the Medtronic® SynchroMed Infusion System®, or am osmotic pump. Systems for monitoring heart function may be directly or indirectly (via wireless technology) coupled to the drug pump.

The present invention and/or one or more portions thereof may be implemented in hardware or software, or a combination of both. For example, the functions described herein may be designed in conformance with the principles set forth herein and implemented as one or more integrated circuits using a suitable processing technology, e.g., CMOS. As another example, the present invention may be implemented using one or more computer programs executing on programmable computers, such as computers that include, for example, processing capabilities, data storage (e.g., volatile and nonvolatile memory and/or storage elements), input devices, and output devices. Program code and/or logic described herein are applied to input data to perform functionality described herein and generate desired output information. The output information may be applied as an input to one or more other devices and/or processes, in a known fashion. Any program used to implement the present invention may be provided in a high level procedural and/or object orientated programming language to communicate with a computer system. Further, programs may be implemented in assembly or machine language. In any case, the language may be a compiled or interpreted language. Any such computer programs may preferably be stored on a storage media or device (e.g., ROM or magnetic disk) readable by a general or special purpose program, computer, or a processor apparatus for configuring and operating the computer when the storage media or device is read by the computer to perform the procedures described herein. The system may also be considered to be implemented as a computer readable storage medium, configured with a computer program, where the storage medium so configured causes the computer to operate in a specific and predefined manner to perform functions described herein. The present invention and/or one or more portions thereof include circuitry that may include a computer system operable to execute software to provide for the determination of a physiological state, e.g., heart failure, bradycardia, etc. Although the circuitry may be implemented using software executable using a computer apparatus, other specialized hardware may also provide the functionality required to provide a user with information as to the physiological state of the individual. As such, the term circuitry as used herein includes specialized hardware in addition to or as an alternative to circuitry such as processors capable of executing various software processes. The computer system may be, for example, any fixed or mobile computer system, e.g., a personal computer or a minicomputer. The exact configuration of the computer system is not limiting and almost any device capable of providing suitable computing capabilities may be used according to the present invention. Further, various peripheral devices, such as a computer display, a mouse, a keyboard, memory, a printer, etc., are contemplated to be used in combination with a processing apparatus in the computer system. In view of the above, it will be readily apparent that the functionality as described herein may be implemented in any manner as would be known to one skilled in the art.

Therapeutically effective concentrations and amounts may be determined for each application herein empirically by testing the compounds in known in vitro and in vivo systems, such as those described herein. Dosages for humans or other animals may then be extrapolated therefrom. With the methods of the present invention, the efficacy of the administration of one or more agents may be assessed by any of a variety of parameters well known in the art.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods. An ion blocking agent of the present invention may be administered at once, or may be divided into a number of smaller doses to be administered at intervals of time.

As used herein, the term “subject” includes, but is not limited to, humans and non-human vertebrates. In preferred embodiments, a subject is a mammal, particularly a human. A subject may be an individual. A subject may be a patient. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

The methods of the present invention include in vitro, ex vivo, and in vivo methods. As used herein “in vitro” is in cell culture and “in vivo” is within the body of a subject.

As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

The present invention includes kits for the delivery of an ion blocking agent. Such kits may include an ion blocking agent and a delivery device for the delivery of the ion blocking agent to a cell or tissue expressing an exogenous polynucleotide encoding a membrane polypeptide that regulates the flow of ions across a cell membrane. Such a kit may also include written direction for use of the kits in methods including, but not limited to, any of the methods described herein.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Modulating Cellular Electrophysiology with Exogenous Ion Channel Expression Can Trigger Unexpected Cellular Remodeling

The expression of exogenous ion channels is an attractive approach to modify cardiac cellular electrophysiology for several translation applications (e.g. arrhythmia and heart failure therapy). It is important to understand what other changes the externally introduced channel might confer to the heterogeneous cell population constituting cardiac tissue. This example uses Kv1.3, a voltage-gated K⁺ channel, as a model system, to determine its effect on cardiac fibroblasts and cardiac myocytes.

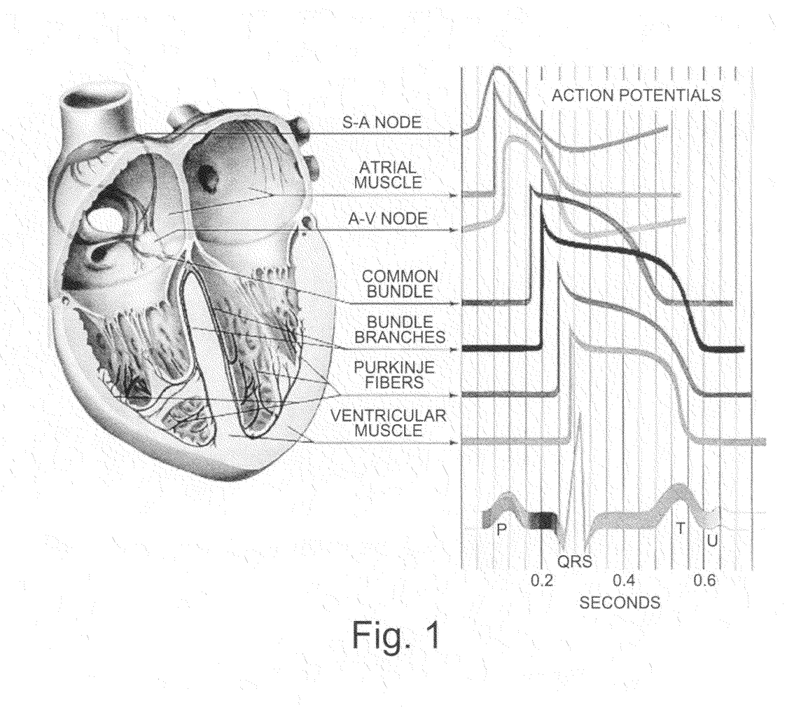

The heart is an electromechanical organ with considerable electrical heterogeneity. This electrical heterogeneity of the heart is conferred by differential expression of ion channels in various regions of the heart (FIG. 1). Several applications are being attempted wherein exogenous ion channels are expressed to change the underlying electrophysiology of the heart.

Ventricular tachycardia, atrial fibrillation, bradycardia, and heart failure all present examples of ion channel therapies for cardiac dysfunctions. As a therapeutic approach in ventricular tachycardia, the over expression of gap junction channels (connexin 43 or other isoforms) may be used to repair conduction in the infarcted regions of the myocardium. Alternatively, remnant connexin 43 may be completely knocked out to eliminate slow conduction (i.e. akin to molecular ablation). As a therapeutic approach in atrial fibrillation, the expression of ion channels may be used to slow conduction in the AV node, thus enabling to control ventricular rate during atrial fibrillation. As a therapeutic approach to bradycardia, expression of exogenous channels (primarily hyperpolarization activated cyclic nucleotide channel; HCN) may induce automaticity in the atrial or ventricular tissue. As a therapeutic approach to heart failure, the expression of SERCA2a may normalize calcium handling in the failing heart.

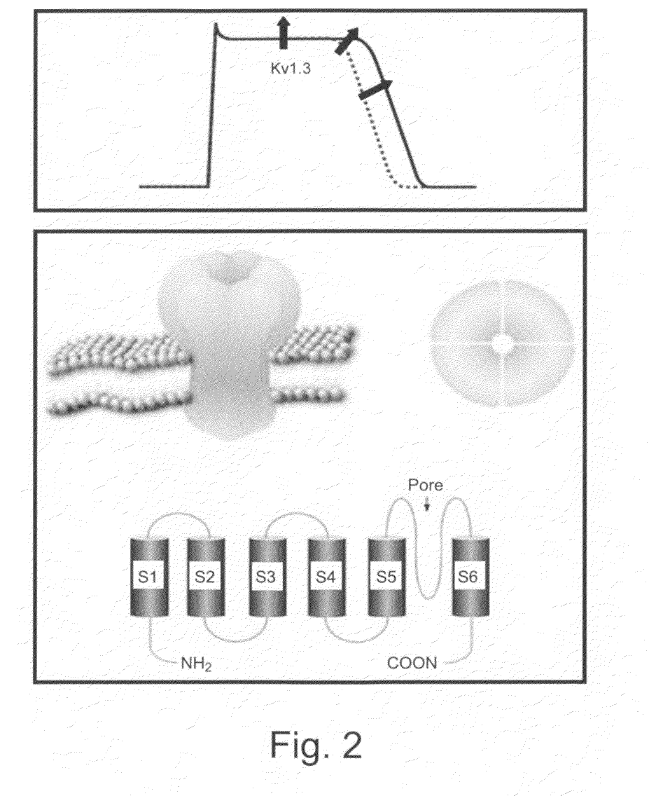

Kv1.3 can serve as a model ion channel in ion channel therapies for cardiac dysfunction. Kv1.3 belongs to the Kv family of channels of which 9 types (Kv1-9) have been described. The Kv1 subfamily has 9 different α-subunits of which Kv1.3 is a member. Ion channels are expressed ubiquitously in both non-excitable and excitable tissues (e.g. expressed in T-lymphocytes, kidney, liver, CNS, skeletal muscle). Kv1.3 is a voltage gated ion channel with rapid activation and very slow inactivation (FIG. 2). Kv1.3 can be inhibited by β-subunit KCNE4. Kv1.3 is small enough to be efficiently packaged into various types of viral vectors.

### Materials and Methods

Cells: Primary neonatal cardiac cells were obtained from ventricles of 2-3-day-old rat pups. The ventricles were fine minced, dissociated in Ca²⁺-free Hank's balanced salt solution (HBSS) supplemented with 0.1%, trypsin and 0.006% pancreatin, and dispersed by triturating.

Separation and culture of cardiac myocytes and fibroblasts: The solution of dispersed cells was supplemented with 10% neonatal calf serum to stop enzymatic activity, centrifuged, resuspended in culture medium, pre-plated in large culture flasks, and incubated for two hours to isolate the fibroblast enriched fraction. Fibroblasts were further purified by media exchange within 1 hour after each passage. The myocytes remaining in suspension were collected and plated at 1.5 10⁵ cells/cm² and grown for five to seven days before experimentation.

Transfection: Subconfluent myocyte and fibroblast cultures were transfected with AdV-Kv1.3-GFP or AdV-GFP at 10⁶-10⁸ PFU/ml. After transfection the cells were cultured in 0-5% serum media with 0, 1, 10 or 100 nM Charybdotoxin (CTx), which is a specific K⁺ channel blocker. After three days in culture, the cells were visually monitored and assayed.

Cell Viability and apoptosis: Cells were assayed for viability and apoptosis with Hoechst nuclei stain.

Immunostaining: Cells were fixed and immunostained for Connexin-43 (Cx43), α-actinin, and vimentin.

### Results

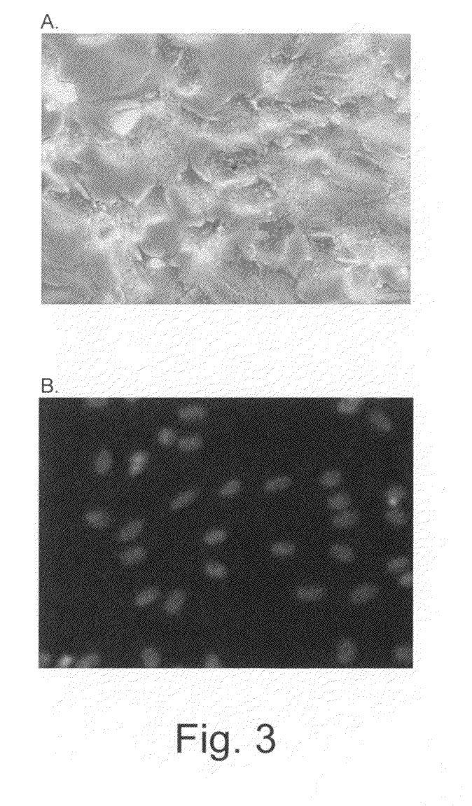

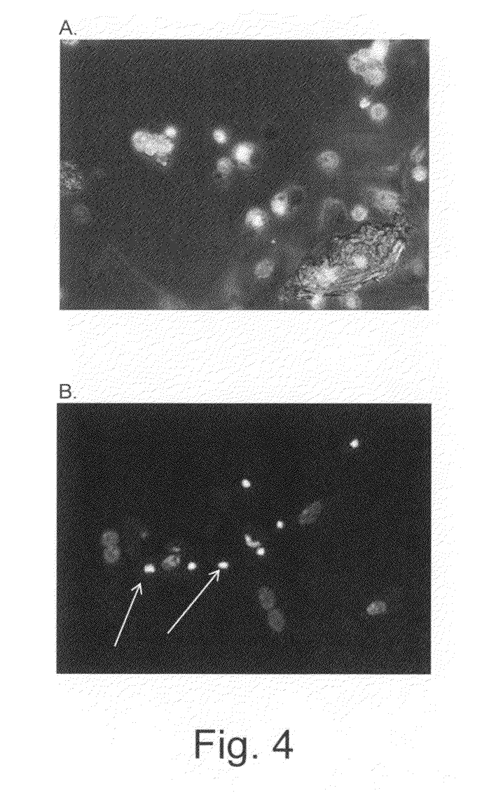

Myocytes and fibroblasts respond differently to Kv1.3 expression: Cardiac myocyte cultures transfected with Kv1.3 stopped spontaneous beating, a result consistent with overexpression of K⁺ channel expression. Myocytes remained viable without significant changes in their morphology (FIG. 3). Myocytes were transfected with 10⁸ PFU/ml AdV-Kv1.3-GFP and brightfield microscopy confirmed expression of Kv1.3 (FIG. 3A). Cell viability is confirmed by Hoechst dye staining of non-apoptotic cell nuclei (FIG. 3B). To the contrary, cardiac fibroblast cultures transfected with Kv1.3 showed visible signs of degradation. Cellular blebbing, rounding and detachment are all clearly visible (FIG. 4). Fibroblasts were transfected with 10⁸ PFU/ml AdV-Kv1.3-GFP and fluorescence microscopy confirmed expression of Kv1.3 (FIG. 4A). The induction of apoptotis is demonstrated by Hoechst dye staining. Punctate DNA within the nuclei of cells (indicated by the arrows) is indicative of apoptosis (FIG. 4B).

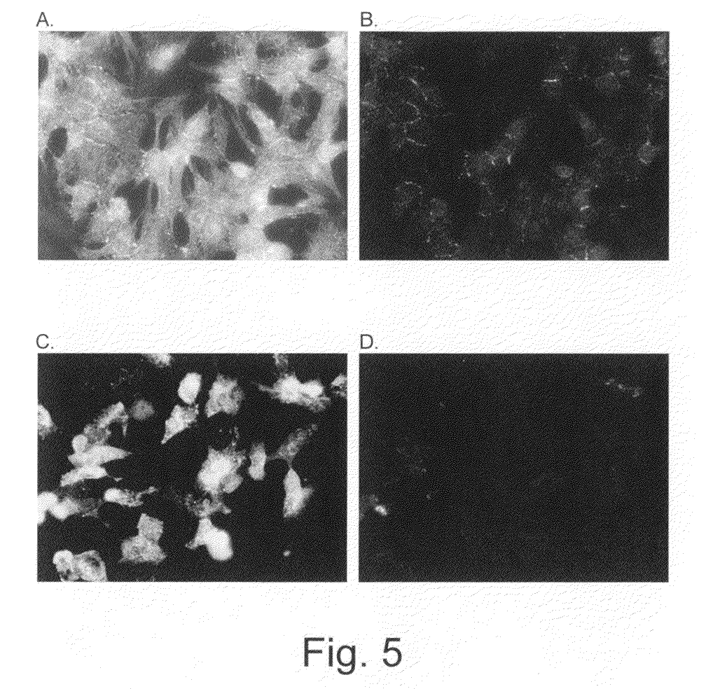

Kv1.3 expression results in reduced Cx43: In Kv1.3 transfected myocytes, immunostaining revealed a dose-dependent reduction in Cx43 expression. Fluorescence microscopy showed expression of both α-actinin and Cx43 in non-transfected cardiac myocytes (FIG. 5A). Myocytes were transfected with 10⁸ PFU/ml Ad-Kv1.3-GFP. Expression of both Kv1.3 and α-actinin was seen in the transfected cells (FIG. 5C). However, Cx43 expression was highly disrupted and down regulated in the transfected myocytes. Fluorescent imaging of Cx43 alone exhibited reduced expression in the transfected cells (FIG. 5D) when compared to imaging of Cx43 alone in the non-transfected cells (FIG. 5B).

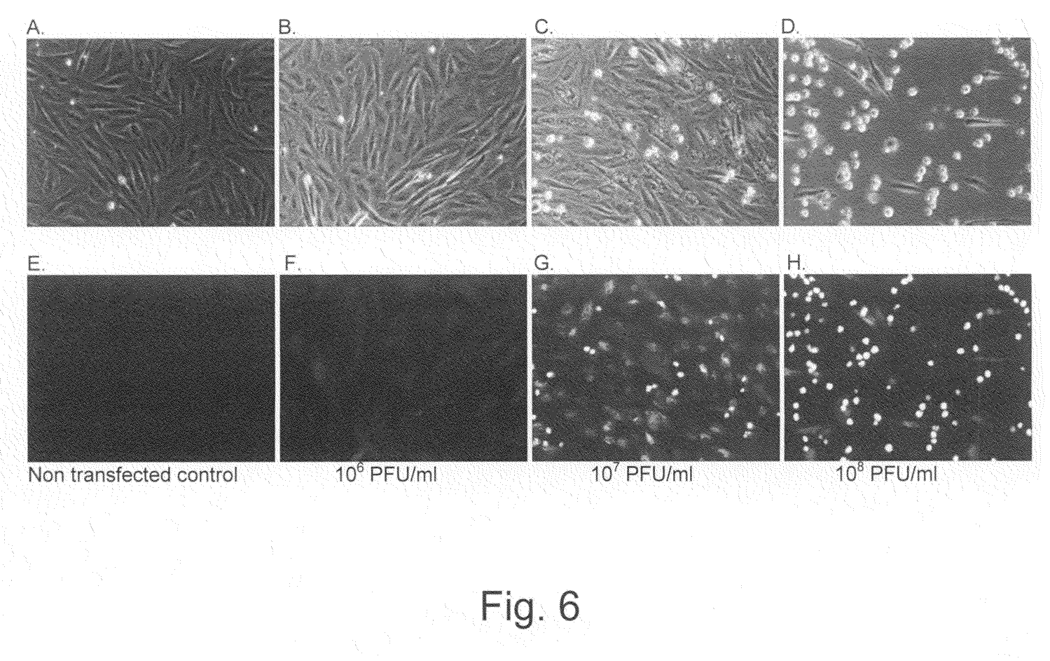

Kv1.3 transfected fibroblasts showed a dose-dependent deterioration: Cardiac fibroblasts transfected with AdV-Kv1.3-GFP demonstrated dose-dependent deterioration. Cells were transfected with 10⁶ PFU/ml AdV-Kv1.3 (FIG. 6B, F), with 10⁷ PFU/ml AdV-Kv1.3 (FIG. 6C, G), with 10⁸ PFU/ml AdV-Kv1.3 (FIG. 6D, H), or left as non-transfected controls (FIG. 6A, E). Phase contrast microscopy showed the changes in cellular morphology (FIG. 6A-D). Fluorescence microscopy confirmed expression of Kv1.3 (FIG. 6E-H). Expression of Kv1.3 results in cellular deterioration in a dose-dependent manner.

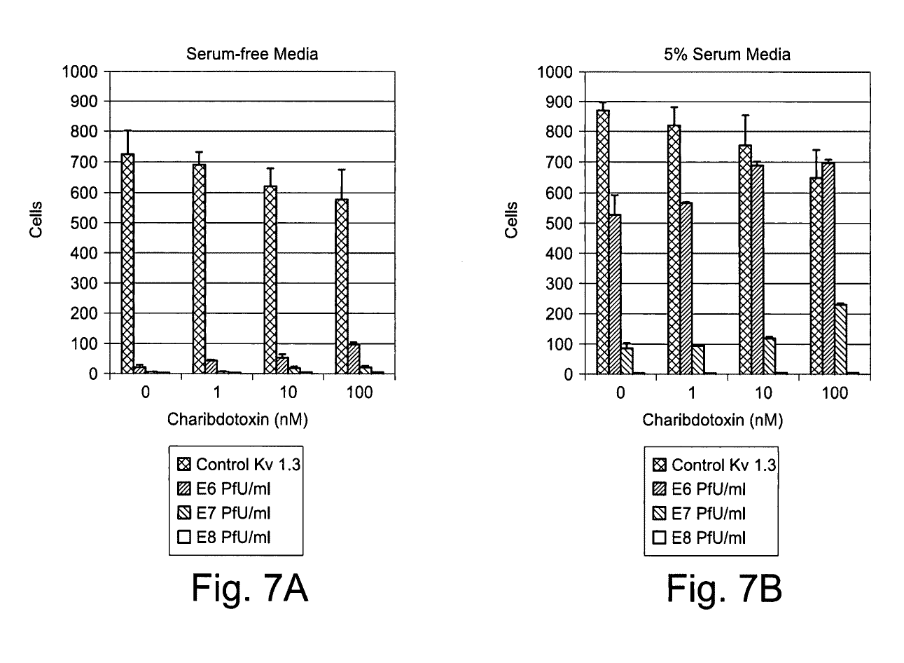

Charybdotoxin rescues Kv1.3 induced cell deterioration: As previously shown, expression of Kv1.3 resulted in cellular deterioration. The addition of Charybdotoxin was able to rescue cells from both apoptosis and morphological degradation. Kv1.3 transfected fibroblasts showed a dose dependent increase in cell apoptosis (n=3 for each titer; FIG. 7). Addition of Charybdotoxin (cTx) alone (FIG. 7A) or addition of cTx and 5% serum (FIG. 7B) partially rescued the cells, thus implicating Kv1.3 as trigger of apoptosis. No changes were observed in control cultures (n=3).

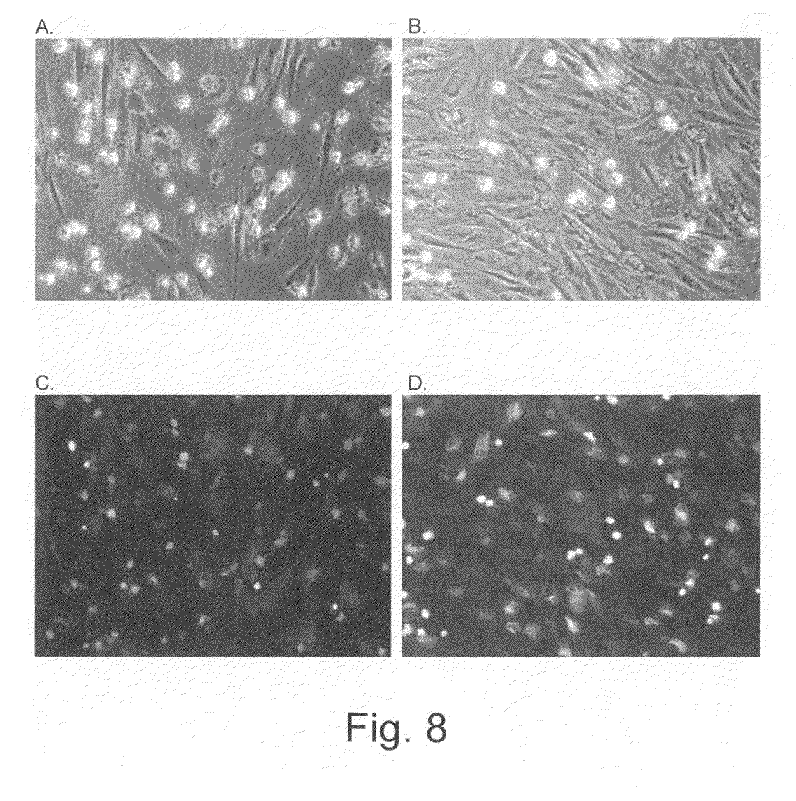

In addition, cardiac fibroblast cell viability was rescued in cells expressing Kv3.1 with the addition of Charybdotoxin. Phase contrast microscopy showed cellular morphology of the fibroblasts. Cells expressing Kv1.3 show cellular degradation. Kv1.3 induced degradation of cardiac fibroblasts by blebbing, rounding, and detachment (FIG. 8A). However, the addition of cTx to cells expressing Kv1.3 maintained normal cellular morphology (FIG. 8B). Fluorescent microscopy confirmed expression of Kv1.3 following transfection with 10⁷ PFU/ml AdV-Kv1.3-GFP (FIG. 8C, 8D).

### Discussion

This example showed that an exogenously delivered ion channel not only produces the intended changes in electrophysiology, but can potentially trigger a range of other molecular remodeling that may be important to consider in translation applications. This example also showed that the addition of the ion channel blocker charybdotoxin can protect cardiac fibroblasts from this deleterious effect. Cardiac fibroblast cell viability was rescued in cells expressing Kv3.1 with the addition of Charybdotoxin.

### Example 2

### In Vivo Transfection with Ion Channel and Administration of Ion Channel Blocking Agent

An ion channel construct, including, but not limited to the AdV-Kv1.3-GFP and AdV-GFP constructs described in Example 1 will be transfected in vivo into the cardiac tissue of an animal, including, for example, a rat or pig. The viability of cardiac myocytes and fibroblasts will be assayed with and without the administration of an ion channel blocking agent, such as, for example, Charybdotoxin.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method of increasing the viability of cells expressing an exogenous polynucleotide encoding a membrane polypeptide that regulates the flow of ions across a cell membrane, the method comprising contacting the cells expressing the exogenous polynucleotide with an ion channel blocking agent, wherein the membrane polypeptide that regulates the flow of ions across a cell membrane is not a hyperpolarized activated cyclic nucleotide (HCN) channel.

2. The method of claim 1, wherein the membrane polypeptide that regulates the flow of ions across a cell membrane is an ion channel.

3. The method of claim 1, comprising contacting the cell with the ion channel blocking agent before, after, and/or coincident to the expression of the polynucleotide encoding the polypeptide.

4. The method of claim 1, wherein the ion channel blocking agent is formulated in composition for controlled or sustained release.

5. The method of claim 1 wherein the cells comprise nonexcitable cells.

6. The method of claim 1 wherein the cells comprise excitable cells.

7. The method of claim 2, wherein the ion channel comprises a gap junction channel, calcium channel, a sodium channel, a chloride channel, SERCA2a, a non-specific leak channel, or a potassium channel.

8. The method of claim 2, wherein the ion channel comprises a potassium channel.

9. The method of claim 8, wherein the potassium channel comprises a member of the Kv1-9 family.

10. The method of claim 8, wherein the potassium channel comprises Kv1.3.

11. The method of claim 1, wherein the ion channel blocking agent comprises a calcium channel blocking agent, a sodium channel blocking agent, a gap junction channel blocking agent, a chloride channel blocking agent, a SERCA2a blocking agent, a non-specific leak channel blocking agent, and/or a potassium channel blocking agent.

12. The method of claim 11, wherein the ion channel blocking agent comprises a potassium channel blocking agent.

13. The method of claim 11, wherein the ion channel blocking agent comprises charybdotoxin.

14. A method of increasing the viability of nonexcitable cells in a heterologous tissue expressing an exogenous polynucleotide encoding a membrane polypeptide that regulates the flow of ions across a cell membrane, the method comprising:
contacting the cells expressing the exogenous polynucleotide with an ion channel blocking agent.

15. A method of increasing the viability of cells in a heterologous tissue expressing an exogenous polynucleotide encoding a membrane polypeptide that regulates the flow of ions across a cell membrane, the method comprising:
contacting the cells expressing the exogenous polynucleotide with an ion channel blocking agent,
wherein the membrane polypeptide that regulates the flow of ions across a cell membrane is not a hyperpolarized activated cyclic nucleotide (HCN) channel.

16. The method of claim 15, wherein the heterologous tissue comprises excitable cells and nonexcitable cells.

17. The method of claim 15, wherein the method increases the viability of nonexcitable cells.

18. The method of claim 15, wherein the method increases the viability of excitable cells.

19. The method of claim 15, wherein the heterologous tissue is cardiac tissue.

20. A method of modifying the electrophysiological function of a heterologous tissue comprising excitable cells and nonexcitable cells, the method comprising:
contacting the heterologous tissue with one or more ion channel blocking agents before, after, and/or coincident to transfection or transduction of the heterologous tissue with an exogenous polynucleotide encoding a membrane polypeptide that regulates the flow of ions across a cell membrane;
wherein the viability of nonexcitable cells in the heterologous tissue is improved.

21. A method of modifying the electrophysiological function of a heterologous tissue comprising excitable cells and nonexcitable cells, the method comprising:
contacting the heterologous tissue with one or more ion channel blocking agents before, after, and/or coincident to transfection or transduction of the heterologous tissue with an exogenous polynucleotide encoding a membrane polypeptide that regulates the flow of ions across a cell membrane;
wherein the membrane polypeptide that regulates the flow of ions across a cell membrane is not a hyperpolarized activated cyclic nucleotide (HCN) channel.

22. A method of modifying the electrophysiological function of a heterologous tissue comprising excitable cells and nonexcitable cells, the method comprising:
transfecting or transducing said heterologous tissue with an exogenous polynucleotide encoding a membrane polypeptide that regulates the flow of ions across a cell membrane; and
contacting said heterologous tissue with an ion channel blocking agent;
wherein the viability of nonexcitable cells in the heterologous tissue is improved.

23. A method of modifying the electrophysiological function of a heterologous tissue comprising excitable cells and nonexcitable cells, the method comprising:
transfecting or transducing said heterologous tissue with an exogenous polynucleotide encoding a membrane polypeptide that regulates the flow of ions across a cell membrane; and
contacting said heterologous tissue with an ion channel blocking agent;
wherein the membrane polypeptide that regulates the flow of ions across a cell membrane is not a hyperpolarized activated cyclic nucleotide (HCN) channel.

24. The method of claim 22 comprising contacting the heterologous tissue with an amount of ion channel blocking agent effective to increase viability of the nonexcitable cells within the heterologous tissue.

25. The method of claim 1, wherein contacting the cells or the heterologous tissue includes intermittent and/or continuous delivery of the ion channel blocking agent.

26. A composition comprising an ion channel blocking agent, wherein the composition is in a formulation for controlled or sustained release.

27. A kit comprising an ion blocking agent and a delivery device for delivery of the ion blocking agent to a cell or tissue expressing an exogenous polynucleotide encoding a membrane polypeptide that regulates the flow of ions across a cell membrane.
